(19) United States
(12) Patent Application Publication
(10) Pub. No.: US 2007/0276286 A1
(43) Pub. Date: Nov. 29, 2007

(54) Device for Tissue Diagnosis and Spatial Tissue Mapping

(76) Inventor: Craig James Miller, Portland, OR (US)

Correspondence Address: Craig Miller, 1209 SW 6th Ave, Apt 505, Portland, OR, 97204, US

(21) Appl. No.: 11/420,731

(22) Filed: May 27, 2006

(51) Int. Cl. A61B 10/00

(52) U.S. Cl. 600/564

(57) ABSTRACT

A miniature electrode array is utilized to stimulate tissue and measure tissue response. When the tissue response is used to diagnose tissue, the spacing of the electrodes in the array is approximately equal to the depth of tissue which is to be examined. The manufacture of such arrays can be accomplished by embedding printed circuit boards, for example, into a properly shaped probe tip. A kinetic device to position the probe on the surface of the tissue provides location information which is correlated with the simultaneous electrical response data in order to generate a tissue map. The tissue mapping capability can be combined with tissue removal devices in order to direct the excision of tissue. When the tissue response is used simply to determine that the array is in contact with the tissue, the electrodes can be widely spaced. Combined with a cell collection device, the electrodes provide feedback to the practitioner that the collection device is making proper contact over the region where cells are to be collected.

### CROSS REFERENCE TO RELATED APPLICATIONS

Provisional Patent Application

U.S. Application No. 60/684,881

Filing Date: May 26, 2005

Name of Applicant: Craig James Miller

Title of Invention: Cervical Bioimpedance Probe

### FEDERALLY SPONSORED RESEARCH

Not Applicable

### SEQUENCE LISTING OR PROGRAM

Not Applicable

### BACKGROUND OF THE INVENTION

1. Field of the Invention

This invention is intended for use in the diagnosis of tissue types of human and animal subjects. It performs electrical measurements as it is moved over the surface of the tissue and from these measurements creates maps of tissue on and below the surface. The invention can also use the electrical measurements to aid in collecting cells, sampling tissue, or treating tissue.

2. Prior Art

(a) Many patents have been lodged that employ electrical measurements on tissue to arrive at a diagnosis. Such diagnoses typically relate to the detection of cancerous or precancerous tissue. For the most part previous attempts to use electrical measurements for tissue diagnosis have suffered from low performance as measured by their combined sensitivity, which is the ability to correctly identify abnormal tissue, and specificity, which is the ability to correctly identify normal tissue. Previous workers who have attempted to use electrical impedance of tissue for the detection of cancer near the surface have not been successful because they have primarily measured the bulk impedance of tissue averaged over a depth far exceeding the region of interest.

(b) Existing techniques for cell collection by scraping, abrading, or otherwise removing surface cells for subsequent laboratory examination have no feedback mechanism to indicate thoroughness in collecting samples over an entire region. For example, scrapers or brushes used to collect cell samples for the Pap test often do not sample the cervical canal and the user of the scraper or brush has no indication at the time of the cell collection whether the user has sampled the canal and has sampled cells over the entire surface of the cervix.

(c) Existing techniques for obtaining deeper tissue samples below the surface using a punch or scalpel type instrument are guided visually and select areas for sample collection using only visual surface cues. The positioning of the tissue removal instrument is difficult and inexact because it is hand-guided.

(d) Surface cell collection using scrapers, brushes, abrading instruments or the like do not record the specific location where the cells were collected. If subsequent laboratory examination of the cells identifies specific cell types, the extent of these cell types is unknown. If one examination shows abnormal tissue and a repeat examination does not, it is not possible to know if the abnormal tissue has repaired itself or whether the repeat examination simply missed the initial abnormal tissue area. Abnormal cervical tissue very often regresses to normal tissue by natural healing processes. In the case of cervical tissue changes, disease is slow to develop, usually taking more than ten years to progress from initial abnormalities to invasive cancer. Because the location and extent of the abnormality are unknown using current cell collection methods, these natural tissue repair processes cannot be monitored except by expensive diagnostic procedures. Therefore, practitioners find it safer to overtreat abnormal tissue by removing it instead of monitoring its progression. In the case of cervical tissue removal, such surgery can affect fertility.

(e) The current cell collection and laboratory examination for cervical abnormalities using the Pap test generates 2 million ambiguous results each year in the United States. This leads to repeat examinations and expensive sophisticated diagnostic procedures.

(f) Current cell collection techniques require subsequent laboratory analysis which generally takes weeks. No immediate results at the time of examination are available to the patient. This produces patient anxiety and creates the opportunity for laboratory data mix-ups in patient records. Patient follow-up can be difficult because patients must be contacted and scheduled for subsequent retesting or further diagnostic work. The delay in laboratory processing is a large problem for migrant communities and underdeveloped areas where social and economic issues limit the opportunities for scheduling multiple medical visits.

(g) For a medical test using cell collection and subsequent laboratory analysis, the microscopic cell analysis depends on operator proficiency and judgement. In particular, the evaluation of the cells collected for a Pap test is subjective and the analysis of a single microscopic slide containing possibly abnormal cells varies between operators and between laboratories.

(h) In existing electrical tissue probes, the electrodes are positioned manually and no position data is obtained. Each location tested by such methods is uncorrelated to other location readings.

(i) For precancer and cancer of the cervix, lesions begin first beneath the surface and then develop outward towards the surface. Surface cell collection often does not detect the initial deeper tissue changes.

### OBJECTS AND ADVANTAGES

Several objects and advantages of the present invention are the following:

(a) The invention restricts the penetration depth of the tissue-stimulating electrical signals through the use of miniature electrodes. This approach is effective because the depth of penetration of electrical signals that probe the tissue is primarily determined by the distance separating the electrodes, the depth of penetration being approximately equal to the electrode separation distance. This remedies the deficiencies of previous systems which sampled bulk properties of tissue well below the depth of interest.

(b) When a cell collection device is intended to be in contact with tissue and collecting cells, simple electrical measurements made with the invention can be used to determine whether the cell collection device is actually in contact with the tissue. Coupled with a scraper, brush, or other surface cell collection device, these measurements of the invention provide feedback to the user that ensures thorough cell collection throughout the region of interest. For a specific tissue examination, such as the Pap test, the invention provides immediate feedback to guide the examiner in collecting cell samples completely over a region of interest, such as the entire surface of the cervix and the cervical canal. The invention will thus reduce sampling errors by controlling the cell collection procedure. For the Pap test cell collection, the invention will continuously signal the provider that proper contact is being made as the scraper or brush or other collection device is rotated over the cervix.

(c) Techniques for obtaining deeper tissue samples below the surface using a punch or scalpel type instrument can be guided by the electrodes of the invention to target areas for sample collection precisely. The positioning of the tissue removal instrument can be controlled by the electrical tissue analysis information and by the motorized positioning controls connected to the electrical probe tip.

(d) The device can be used to immediately provide direct tissue diagnosis at each location where the probe is positioned. This detailed analysis of the tissue combined with simultaneous position data will generate a map of normal and abnormal tissue to be used for further diagnostic procedures, to direct treatment so that only abnormal tissue is targeted for removal, or to record and visually display tissue diagnosis so that the practitioner can follow up suspect areas and monitor tissue changes over time. Normal tissue repair processes by the body can be tracked and surgical intervention minimized.

(e) The tissue maps created by the invention will improve accuracy of the tissue analysis by constraining the local tissue analysis to be consistent with neighboring tissue analysis. The invention will utilize both adjacent surface data and adjacent layer data to refine the tissue diagnosis. The tissue map of the entire area of interest will provide a measurement of the extent of tissue changes which will further assist in determining the subsequent examination and treatment.

(f) The invention will provide immediate results to the patient at the time of examination. This reduces patient anxiety and eliminates the opportunity for laboratory data mix-ups in patient records. Patient follow-up can be immediate and will not depend on opportunities for scheduling multiple medical visits.

(g) The invention will provide a non-subjective evaluation of tissue health which does not vary significantly between laboratory technicians or medical practitioners.

(h) The invention contains many electrode pairs. For each position on the tissue, several electrodes provide readings. The position of each of these electrodes is controlled by a positioning device so that all tissue tested can be mapped and correlated. The multiple electrodes on each probe and the automatic positioning allow large amounts of data to be gathered quickly.

(i) The invention controls the depth of tissue analysis and thus allows earlier detection of changes which begin below the surface, improving the quality and timeliness of the diagnosis.

Further objects and advantages will be apparent from a consideration of the ensuing description and drawings.

### SUMMARY

The invention consists of an array of miniature electrodes finely spaced so as to analyse tissue by electrical stimulus and response at controlled depths. The invention uses mechanical devices to position the placement of the electrode array so that the location of all tissue readings is collected and correlated to generate diagnostic tissue maps. The tip containing the electrode arrays can incorporate other devices for cell and tissue collection or for tissue treatment using the position information and the diagnostic analysis of the tissue at each location.

### DRAWINGS—FIGURES

FIG. 1 shows the preferred shape for the cervical tissue probe tip.

FIG. 2 shows one side of a printed circuit board with conducting paths laid out on the PCB and with an edge shaped to match the contour of the tip shown in FIG. 1.

FIG. 3 shows the reverse side of the printed circuit board shown in FIG. 2.

FIG. 4 shows two printed circuit boards as shown in FIGS. 2 and 3 together with a separator before being juxtaposed to fit into a slot in a tip such as that shown in FIG. 1.

FIG. 5 shows two printed circuit boards as shown in FIGS. 2 and 3 together with a separator after being juxtaposed and trimmed to fit into a slot in a tip such as that shown in FIG. 1.

FIG. 6 shows a cervical tissue probe tip shaped as in FIG. 1 and incorporating a brush for cell collection and printed circuit boards as shown in FIG. 5 with the edges of the conductors on the circuit boards serving as electrodes.

FIG. 7 shows a cervical tissue probe tip as shown in FIG. 6 with a connecting shaft to rotate the tip in order to collect cell samples.

FIG. 8 shows a cervical tissue probe tip shaped as in FIG. 1 and incorporating a brush for cell collection and embedded metal conductors serving as electrodes.

FIG. 9 shows a cervical tissue probe tip as shown in FIG. 7 as seen from the bottom.

FIG. 10 shows a cervical tissue probe tip shaped like a paddle and incorporating a brush for cell collection and embedded metal conductors serving as electrodes.

### REFERENCE NUMERALS

100 tip shape for cervical probe
110 substrate for printed circuit board
112 metal electrical conductor of printed circuit board comprising electrode
114 metal electrical conductor of printed circuit board comprising electrical track
116 through hole in printed circuit board to connect to conductor on back side
118 metal pad to connect back side artwork to edge connector
120 metal pad to connect front side artwork to edge connector
122 metal electrical conductor comprising electrode on back side of board
124 metal electrical conductor comprising electrical path on back side of board
130 printed circuit board containing electrodes, conducting tracks, and pads for connection to external cabling
132 spacer to separate printed circuit boards 130 and 134
134 printed circuit board which is a mirror image of printed circuit board 130
136 trimmed section at top of printed circuit board
140 rows of brush fibers for tissue collection
142 electrode rows created by edges of assembled printed circuit boards 130 and 134 and intervening spacer 132
150 shaft to mechanically link probe tip with user or mechanical positioning device
160 wire embedded in probe tip to form electrode at surface of probe tip
170 pocket molded in back side of tip to allow interconnect of wires to standard electrical jack

### DETAILED DESCRIPTION

### Preferred Embodiment

The system consists of (a) a probe tip which contains an array of electrodes and contacts the tissue directly; (b) a handpiece which is mechanically and electrically connected to the probe tip and which consists of a connecting drive shaft assembly, motors or other kinetic devices to position the probe tip precisely, and an electrical connection to the electrodes in the tip; (c) an electrical signal generation device to stimulate the tissue by means of electrical waveforms; (d) a data acquisition device to measure the electrical signal response from the tissue; (e) a processor which controls the signal generation device, data acquisition device, signal response storage and analysis, and the motors or other kinetic devices used to move the probe tip. The signal generation device and data acquisition device may be contained as electronic components and circuits within the handpiece or externally to the handpiece, as in a circuit board located within a computer. Likewise, the electronics used to drive the positioning elements and the electronics used to control the switching that selects which electrodes are active may be within the handpiece or located externally to the handpiece. The handpiece itself could contain multiplexers to control the switching of the electrodes and thus reduce the cabling requirements between the handpiece and an externally located signal generation and data acquisition components. The method of electrical signal generation, data acquisition, and positioning device control are well known to those skilled in the art. The handpiece may communicate with an external processor using a wire cable or through a wireless channel. The handpiece may also be configured so that it operates without connection to a computer. In such a configuration, the handpiece would contains circuitry adequate to signal the operator regarding positioning the probe tip so as to ensure proper contact with the tissue. Such a handpiece would also contain circuitry for electrical signal generation to stimulate the tissue, a data acquisition device to sample the electrical signal response, and memory to store the tissue response waveforms. The tissue analysis could then be performed by circuitry directly incorporated in the handpiece or processed when the handpiece was returned to a docking cradle which communicated with the computer. The tissue analysis could also be performed when a memory device with the tissue response and tissue location data was removed from the handpiece and connected to the computer. Long-term storage of the tissue response and location of the tissue could be stored in order to track tissue changes with time.

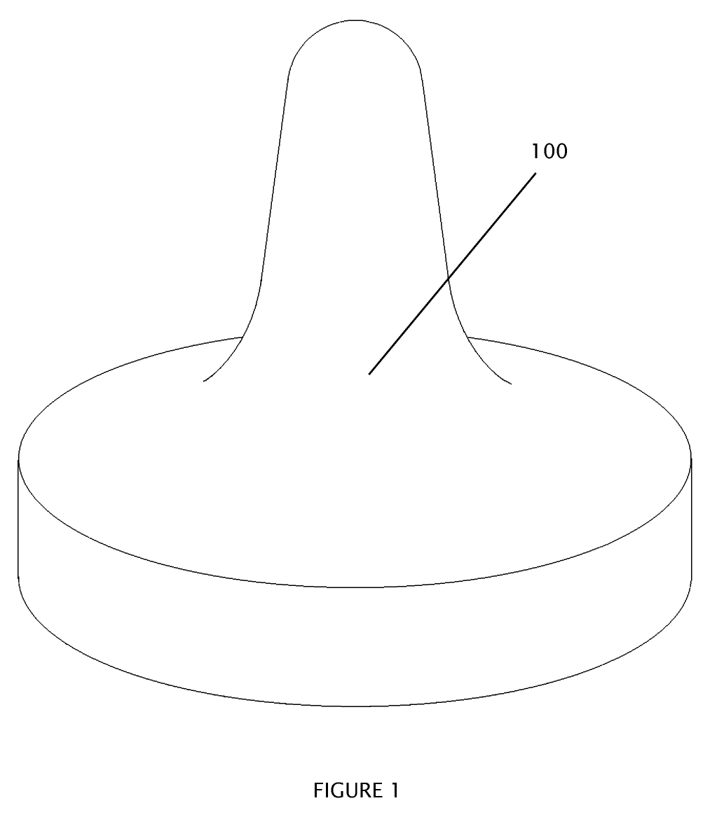

The preferred embodiment for a cervical tissue examination device is illustrated in FIGS. 1-7.

FIG. 1 illustrates the top view of the three-dimensional shape 100 of the probe tip contoured so as to enter the cervical canal and make contact with the external cervix along the rest of the top face.

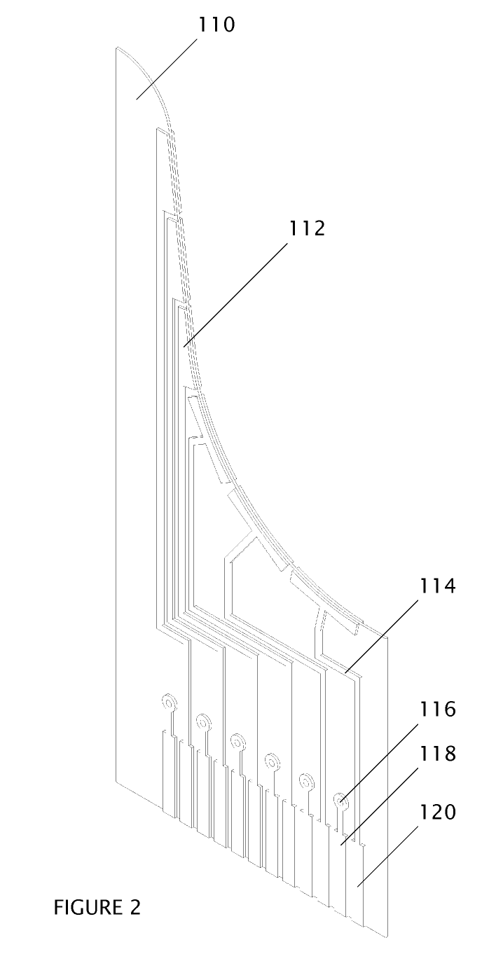

FIG. 2 shows a printed circuit board which consists of a substrate 110 and metal conductors on the surface. The conductors terminate along the contoured edge in pads 112 whose edges on the contour become the electrodes when the circuit board is embedded in plastic. The conducting paths 114 connect the electrode pads to pads 120 on the card edge which are used to connect to standard card edge connectors. Through holes 116 in the substrate connect conducting paths on the back side of the circuit board to pads 118 on the front side again to connect with standard card edge connectors.

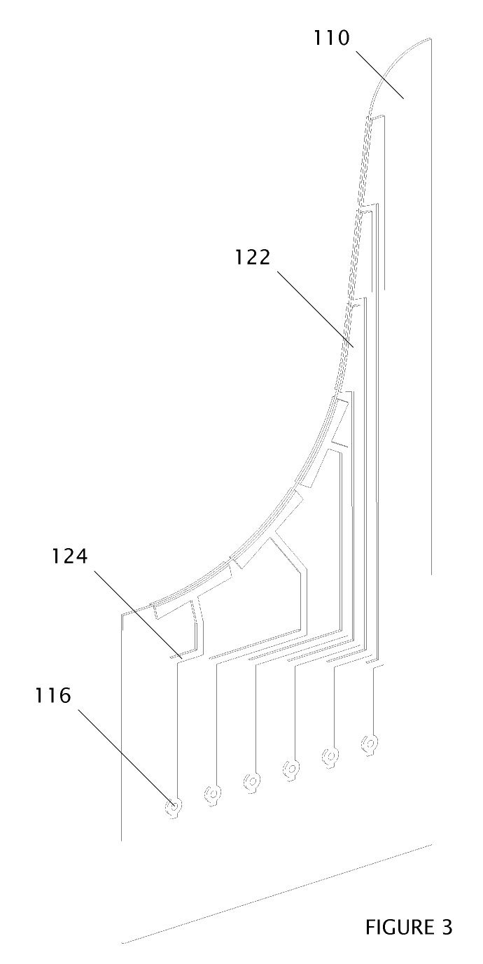

FIG. 3 shows a the reverse side of the printed circuit board in FIG. 2 and consists of a substrate 110 and metal conductors on the surface. The conductors terminate along the contoured edge in pads 122 whose edges on the contour become the electrodes when the circuit board is embedded in plastic. The conducting paths 124 connect the electrode pads to through holes 116 in the substrate as described in FIG. 2.

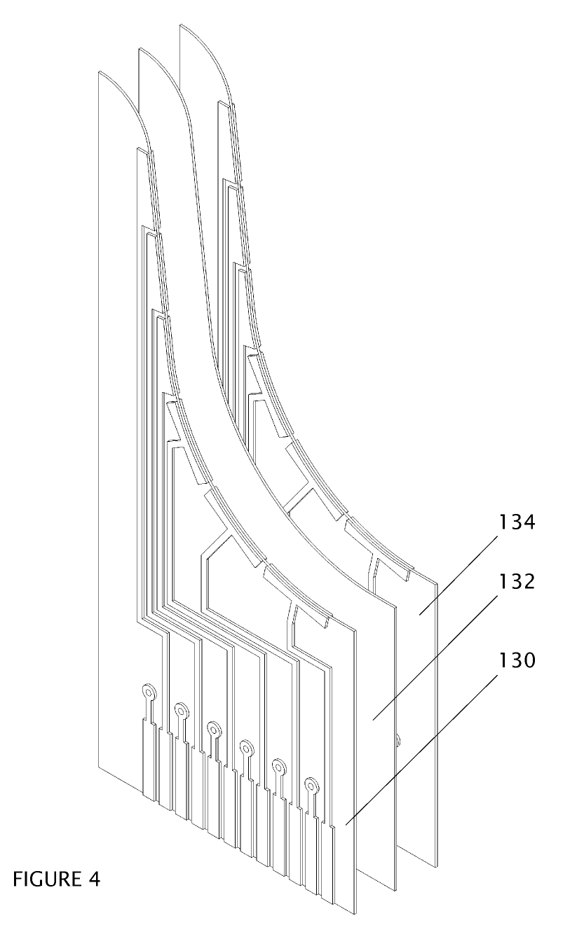

FIG. 4 shows a spacer 132 between two printed circuit boards 130 and 134 as described in FIGS. 2 and 3.

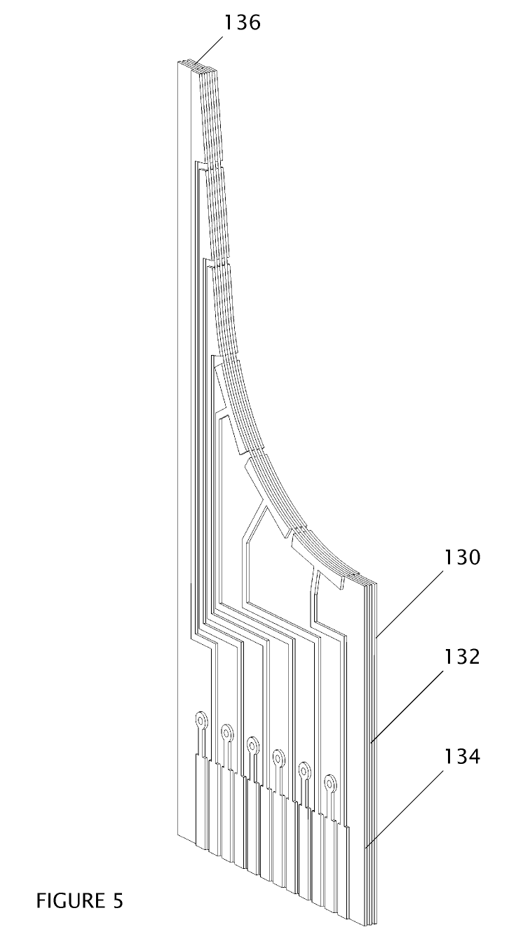

FIG. 5 shows the spacer 132 and circuit boards 130 and 134 juxtaposed and trimmed on surface 136 so as to be inserted into a probe tip shaped as in FIG. 1.

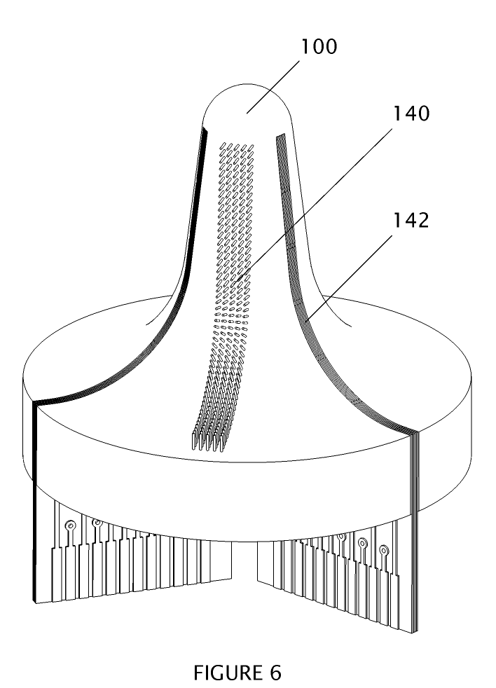

FIG. 6 shows two such assemblies 142 embedded in a probe tip 100 and also containing a row of brush bristles 140 for surface cell collection.

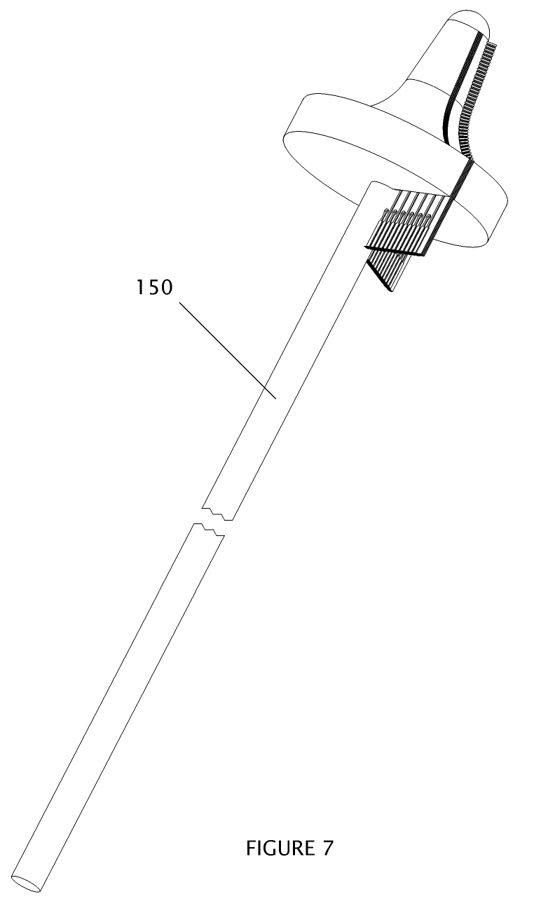

FIG. 7 shows the assembly of FIG. 6 with a mechanical connecting rod 150.

The probe consists of a multitude of electrodes, preferably arranged in parallel rows with minimal separation between the rows and minimal width of the electrode in the row as measured perpendicular to the row. In the preferred embodiment, the tissue is stimulated by custom waveforms consisting of a superposition of sine waves of various frequencies, selected from a range extending from about 50 Hertz to about 500,000 Hertz, each frequency component having its own individual magnitude and relative phase. These composite waveforms are selected to maximize discrimination between various tissue types. Because the depth of probing by a stimulating electrode pair is approximately equal to the distance separating the electrode pair, the individual electrodes are arranged so as to obtain separations less than or equal to the depth of interest in the tissue. For cervical tissue, disease takes place in the first 10-20 cells or approximately the first 300 microns of tissue depth. The separation of the electrodes must range from about 50 microns to 300 microns to avoid sampling bulk tissue information below the depth of interest. The electrodes themselves must be thin or the farthest edges of two parallel electrodes could be separated by distances greater than 300 microns and would sample irrelevant deep tissue. For cervical tissue analysis, the narrow electrodes of this invention have a width on the order of 30-70 microns so that the tissue sampling between two parallel electrodes takes place at a narrow range of depth. Again, for cervical tissue analysis, the narrow electrodes of this invention have a separation ranging from about 50 microns to 250 microns. The length of the electrodes is not as critical and is limited so as to provide detailed sampling of tissue in a small region. The electrodes are long enough to avoid excessive noise from sampling too small a region of tissue. For cervical tissue analysis, the preferred length is on the order of one millimeter. In the preferred embodiment of this invention, the composite waveforms are also customized for each electrode separation, namely, each depth of tissue being probed.

Initial waveforms applied to the tissue may be customized differently than the waveforms used to stimulate the tissue for the purpose of tissue analysis. For example, the initial waveforms may be selected for the following purposes:

(a) to rapidly set up a stable well-understood interface between an electrode and the tissue surface, the interface commonly being known as the double layer;

(b) to load the electrodes with appropriate proteins present on mucosal tissue, such as the cervix, so as to stabilize the electrode characteristics;

(c) to cause electroporation, namely, to stimulate the cells of the cervix so as to allow materials within the cells to pass out of the cell easily.

Different waveforms may also be applied in stages in order to measure different properties of the tissue. Several customized waveforms may thus be applied in testing at a particular location on the cervix.

In the preferred embodiment for examining cervical tissue, the shape of the probe tip is illustrated in FIG. 1. This shape 100 is similar to the shape of a cryotip which is used to freeze diseased cervical tissue for the purpose of removing abnormal tissue by ablation. The cervix is shaped like a torus with a narrow opening in the center. The shape 100 for the probe tip of this invention does not make proper electrical contact with the cervix unless it is inserted into the cervical canal. Together with the electrode feedback which measures tissue contact, this shape ensures that the cervical canal is probed. This remedies failures of the current Pap test method where the cervical canal is often not sampled.

In the preferred embodiment for examination of the cervix, the probe tip has a diameter of about 20 millimeters, somewhat smaller than the diameter of the cervix. The spacing, electrode size, and frequencies used by the invention are suitable for detecting structures down to about 300 microns below the surface of the cervical tissue. To obtain tissue information within that superficial layer of 300 microns, the device stimulates the tissue using narrow electrodes with separations between electrodes being approximately 50 and 250 microns. The probe tip of the device is rotated manually or under motor control in order to test all tissue locations on the cervix. Using a small stepper motor to rotate the probe tip would allow all areas of the cervix to be automatically tested and the known position of each reading simultaneously recorded. A typical electrode configuration consists of four to six electrodes per row, each electrode about one to two millimeters long and 35-70 microns thick. The gap between the electrodes along a row is typically minimal, about 200 microns.

In the preferred embodiment, the electrodes are created on a printed circuit board (PCB) as shown in FIGS. 2 and 3. Using typical PCB fabrication techniques typically produces a base copper track thickness of 35, 70, or 105 microns. This would result in a probe tip electrode thickness of 35, 70, or 105 microns respectively. The PCB can also hold integrated circuit chips such as multiplexers which could be used to minimize the cabling requirements between the probe tip and the handpiece. The PCB can terminate on the rear side, away from the electrode face, in a connector strip 118 and 120 as used for an edge connector, or in a jack or plug attached to the PCB using standard PCB fabrication and assembly techniques. The PCB is then cast into a suitable medical grade resin, such as polystyrene or polycarbonate so that the conducting copper tracks on the PCB lie perpendicular to the surface of the probe tip which contacts the cervical tissue. The thickness of copper laid down on the PCB then becomes the width of the electrode. Parallel electrode pairs are formed by placing thin insulating sheet approximately 50-200 microns thick between pairs of parallel PCBs as shown in FIGS. 4 and 5. The parallel PCBs could also be spaced apart in the mold and separated by the resin filling the mold, thus creating the insulating layer between the electrodes. The cast product can be molded directly to final shape or be cut to final shape on a lathe or by a milling machine. FIG. 6 shows PCB assemblies from FIG. 5 embedded in a probe tip shaped to fit the cervix and cervical canal. FIG. 6 also shows a row of brush bristles which can optionally be embedded into the tip between the electrode rows. In such an application, tissue contact is measured electrically by the electrodes as cells are collected by the brush. Multilayered PCBs could also be used to create parallel rows of thin electrode pairs where the stacking separation of the layers within the PCB itself governs the electrode separation distance. Alternately, electrode spacing could be controlled on a two-sided board by the thickness of the board itself.

To fabricate the miniature electrode array, the insulating substrate 110 of the PCB is coated with copper alloy in the usual method for circuit boards. The patterns of the electrodes 112 and tracks 114 are manufactured as with printed circuit boards or cut with laser tools, for example. The method of manufacturing finely spaced and precisely located conducting tracks with closely controlled conductor thickness on a PCB is well known to those skilled in the art. The electrodes on the tissue end of the probe can be parallel from row to row or offset half the length of an electrode as is sometimes done in bioimpedance applications. Several electrodes can be connected simultaneously to the signal generating and response measuring equipment in order to produce different effective electrode shapes. Bipolar and more noise-tolerant quadrapolar measurements are possible by connecting four electrodes in pairs.

Protein coatings such as amino acids or mercaptans may be used to normalize the electrode properties independent of the conductive structure beneath the coating. The electrodes themselves may be composed of a metal, a semiconductor, a conducting polymer, or other conductor. The coating ensures that a stable, predictable electrochemical interface is created between the probe tip electrodes and the tissue. This interface is referred to in the literature as the double layer.

Different coatings on different electrodes of a single probe can be used for different functions, such as pH measurement, detection of optimal contact, or detection of material adsorbed to the electrode, such as proteins or other biological materials. The coatings could be used to detect patient antibodies or other biological system markers not necessarily directly related to the tissue condition of the cervix itself, such as the status of an embryo in the uterus, hormone levels, or other proteins, biological by-products, or contaminants that are suitable for sampling at the cervix. For the most effective sampling of material contained within the tissue cells, stimulating waveforms may be applied to make the cell membrane porous to these inclusions of the cells. This technique of electroporation is well known to those skilled in the art.

In the preferred embodiment for examination of the cervix, the handpiece has a motor that rotates the probe tip connecting shaft 150. The advantage in contacting the cervix in a controlled manner using precise mechanical positioners is that the cervix is not only sampled comprehensively and a map of the cervix obtained, but that the information from neighboring tissue on the surface and below the surface can be used to refine the analysis of tissue.

To ensure single use of the device, a miniature fusible link or similar circuit element can be incorporated onto the tip circuit board so that at the start of the probing procedure, this link is tested for continuity in order to proceed. After passing this test, the device sends a signal to this link to burn it out, thus ensuring that the tip is not reused.

### Additional Embodiments

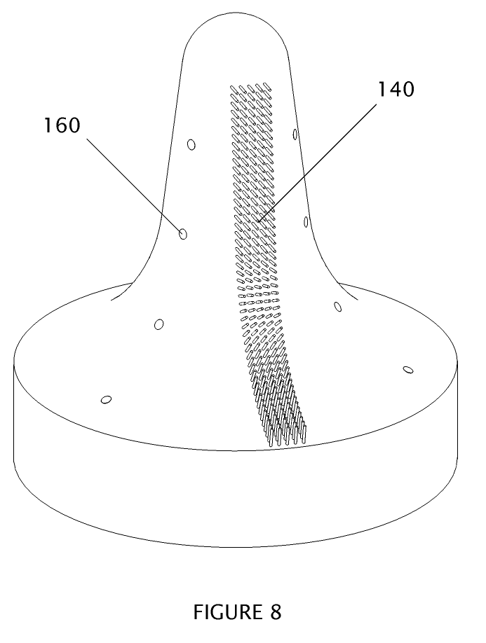

FIG. 8 shows the top view of a probe tip 100 with embedded wires 160 forming the electrodes as they terminate on the surface of the probe contour. The tip also incorporates a row of brush bristles 140 for surface cell collection.

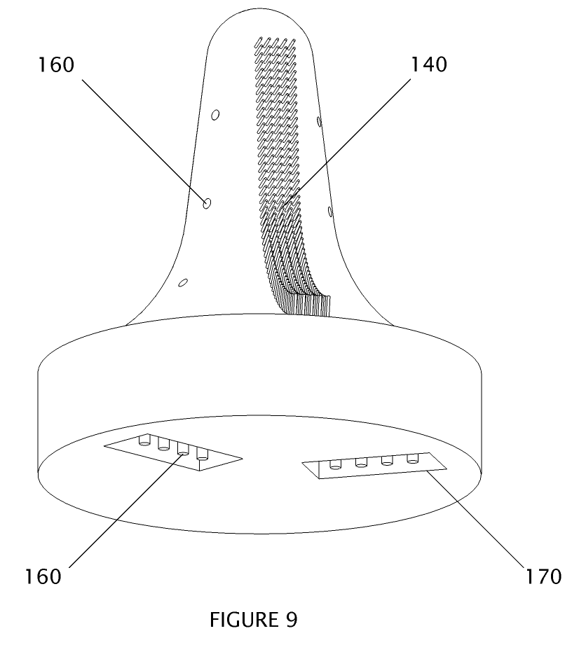

FIG. 9 shows a bottom view of the probe tip shown in FIG. 8. The bottom side has molded pockets 170 and wires 160 terminating in an arrangement so as to mate with standard electrical jack connectors.

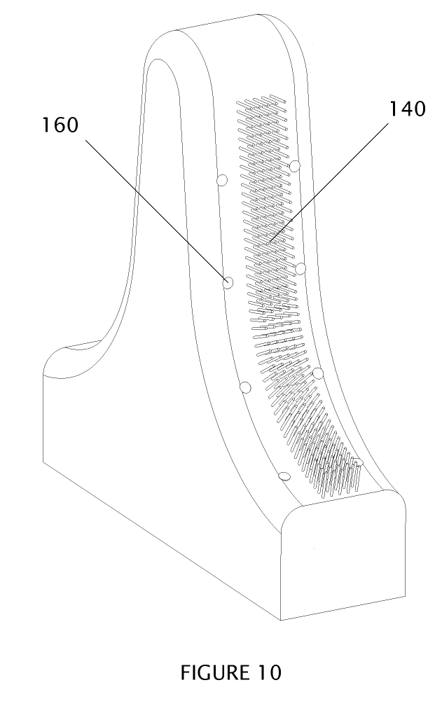

FIG. 10 shows a paddle with cross-sectional contour similar to the contour of FIG. 1. The paddle incorporates wires 160 forming the electrodes as they terminate on the surface of the probe contour. The tip also incorporates a row of brush bristles 140 for surface cell collection.

Electrodes may created by embedding wire of various shapes into the probe tip and molding around the wire. Round wire 160 embedded in a probe for cervical cell collection is shown in FIG. 8. The brushes 140 collect the cell samples and the electrodes 160 measure electrical contact as the tip is rotated to collect cell samples. FIG. 9 shows the wire electrodes 160 for this type of probe terminating on the back side in a pocket 170 with spacing suitable for standard cable connectors.

For cell collection devices, the collector may be a scraper, a brush, an abrader of another sort such as a ribbed or bumped surface, or various adhesive materials to which the cells will adhere, such as hydrogels.

Other methods of creating electrodes on thin film may be employed. These include (a) molded electrodes and connecting tracks using conducting plastics, (b) conducting inserts molded directly into the plastic probe tip, (c) deposition of metals, semiconductors, or other conducting material onto films, sheets, or cross-sections of a probe tip using sputtering, spray metallization, selective plating using lithography, or otherwise depositing conductors into a miniature electrode array in order to measure tissue at shallow, finely controlled depths.

The electrical connection between the probe tip electrodes and the electrical signal generation and measurement components can be wire cable or other conducting paths incorporated in the connecting shaft, for example.

For cervical cell collection, the electrodes can also be embedded in a tip shaped more like a standard cervical scraper of the Rover's type or other paddle-shaped collection devices. FIG. 10 illustrates such an embodiment with embedded wire electrodes 160 placed on the sides of the cell collection brush 140. The electrodes in this embodiment are used to ensure tissue contact over the entire surface of the cervix as the paddle is rotated to collect cell samples.

In another embodiment, the electrodes are simply the outer coatings of optical fibers with a conductive sheath on each fiber. The fiber bundle is heated and drawn as in a fiber taper bundle in order to reduce the size of the connecting bundle and to orient the fibers so that they are nearly perpendicular to the surface of the probe tip. The outer conducting coating on each fiber acts as an electrode at the surface. The fiber bundle can also be used to transmit optical information, namely visual images to guide the operator in locating the endocervical canal, for example, or to record optical images of the cervix or to perform other optical measurements, such as tissue response to various frequencies of light, on cervical tissue or other tissue which is being examined. The optical images could be correlated spatially with the electrical images generated by the stimulus-response of the tissue.

In other embodiments for examining tissue, an optical detector could be incorporated into the device to detect contact with the surface. Such a device could consist of a cell collection component, such as a brush or scraper, and an optical source and detector used to ensure contact. The method of operation could rely on the change in the measured optical properties of the tissue because pressure on the tissue restricts blood into the area of contact. Such a change in optical properties can be easily measured by the change in absorption of red, green, or blue light, for example. Likewise, local pressure-detecting components could be incorporated into a probe tip to ensure that contact is made across the surface of the tissue.

### Advantages

This invention overcomes the limitations of the prior art by making a range of measurements that are controlled as to waveform shape, depth of penetration, and precise mappable location of the tissue. The tissue response is then interpreted to arrive at an accurate diagnosis of the state of the tissue.

The detection system consists of an electrode assembly geometrically suited to the nature of the tissue to be diagnosed and a measuring system comprising appropriate electronic circuits. The features that distinguish this invention from the prior art lie in
1. the type of electrode, including the electrode geometry, method of fabrication, and coatings applied to the electrode surface to facilitate accuracy in tissue diagnosis or to collect other biological cell products;
2. the use of electrical measurements to determine that contact has been made directly with the tissue or at least as near to the tissue surface as is practically possible due to the biological fluids which may normally be present on the tissue;
3. for cervical tissue diagnosis, the use of a probe tip shaped to be inserted into the cervical canal and using the tissue contact measurements to ensure that the canal has been probed;
4. the motorized handpiece which positions the probe tip and correlates electrode position with actual position on the tissue;
5. the mapping of tissue using tissue response data and the position data from the motorized handpiece;
6. the use of the mapping information to enhance tissue analysis using the requirement that tissue readings across the surface of the cervix must be coherent with adjacent readings;
7. the method of stimulating the tissue using composite waveforms consisting of pure sine waves of selected frequencies with different magnitudes and relative phases superposed and constructed so as to maximize tissue response differentiation;
8. the matching of tissue response to tissue type using composite signal pattern matching;
9. in certain tissue applications, such as cervical tissue screening for precancer and cancer, a collection device such as a scraper edge or a brush incorporated into the probe tip that collects tissue continuously as the probe tip is moved across the tissue surface and using electrical contact measurements to ensure that cell samples are collected at all points across the scanned tissue;
10. the incorporation of a tissue sampling device such as a biopsy punch into the probe tip and using electrical stimulus-response measurements to guide the user in collecting tissue samples at locations identified by the tissue analysis software;
11. the incorporation of a tissue treatment component, such as a cryotip or a wire excision instrument, directly into the probe tip so that tissue ablation can be accomplished with fine control using electrical stimulus-response measurements to guide the user in removing all diseased tissue and restricting tissue removal to that tissue specifically targeted;
12. the incorporation of a fusible link in the probe tip in order to ensure single use of the tip;
13. the use of a bar code or other unique identifier for each tip in order to ensure single use of the tip;
14. a fingerprint reader to collect unique identification from the patient to link permanently and uniquely with the data collected so that data loss or crossover is impossible, even if other personal identifier data, such as name or birth date, is not unique or is corrupted in data entry;
15. use of additional patient information correlated with increased risk of cancer, namely, age, history of smoking, years using birth control pills, number of births, presence and history of HPV type, presence of other sexually transmitted diseases, and immune system deficiencies, so that the system could predict individualized patient risk based on the current extent of tissue changes and existing data on the natural history of cancer precursors.

The device employs electrode spacing and frequency variation to control the depth of tissue which is probed. In an application which diagnoses tissue of the cervix, the abnormal tissue originally appears below the surface and develops outward. In such an application, the depth information provided by the invention will provide early detection of changes in the cervix. The invention will better diagnose the state of the tissue than existing methods which employ electrical measurements because it will use the location information for each set of electrical stimulus-response measurements to refine the analysis. For example, tissue types must spatially form coherent transitional boundaries which means the location information can provide a method to clarify readings which are not distinctly identifiable through purely electrical measurements. The tissue type tested electrically in a small section must be coherent with neighboring tissue types in all directions. In particular, for tissue changes of the cervix, the tissue type at a particular location on the surface must be consistent with changes that progress continuously as tissue is sampled below that particular location on the surface.

In the case of cervical tissue abnormalities, most precancerous changes repair themselves over time. The invention can store tissue maps in a three-dimensional graphic data display which will be generated and recorded for each patient. In this manner, the progress of the cervical tissue changes can be monitored over time and tissue which regresses to a normal form need not be removed, thus minimizing surgical intervention.

The algorithms employed for analysing and classifying tissue are derived from data gathered using this device or similar devices. Data from known tissue types are used as standards to which the response is compared and optimally matched.

I claim:

1. a device for diagnosing tissue in situ by electrical stimulus and response measurements by means of miniature electrode arrays embedded in a probe tip.

2. an addition to the device of claim 1 to ensure that the probe tip is not reused by incorporating a fusible link into the probe and burning it out during the first use of the tip.

3. an addition to the device of claim 1 to ensure that the probe tip is not reused by the use of a bar code or other unique identifier for each tip which is recorded and stored;

4. a method of diagnosing tissue accurately by stimulating the tissue using composite waveforms consisting of pure sine waves of selected frequencies with different magnitudes and relative phases superposed and constructed so as to maximize tissue response differentiation and subsequently matching of the tissue response to tissue type using composite signal pattern matching.

5. a device for mapping tissue in situ by using electrical stimulus and response measurements coupled with a positioning device so as to provide simultaneous tissue analysis and tissue location.

6. a method using the device of claim 5 to minimize surgical intervention to remove tissue by allowing the practitioner to monitor tissue changes over time which allows natural healing processes to proceed with minimal risk to the patient.

7. a method using the mapping information of claim 5 to improve the electrical diagnosis of tissue using the requirement that tissue readings across the surface of the cervix must be coherent with adjacent tissue readings.

8. a device to guide the practitioner in precise tissue removal by means of a tissue removal device coupled with an electrode array which diagnoses tissue by electrical stimulus response.

9. a device to ensure tissue surface contact during a cell collection procedure by using electrical measurements performed during the cell collection procedure.

10. the device of claim 9 used to provide continuous auditory feedback to the practitioner performing the cell collection so that the practitioner is notified in real time that the collection device is making proper contact with the tissue.

11. the device of claim 9 used to provide continuous visual feedback to the practitioner performing the cell collection so that the practitioner is notified in real time that the collection device is making proper contact with the tissue.

12. a device that ensures that cells are collected from the cervical canal by means of a collection device shaped to simultaneously fit into the canal and touch the external surface of the cervix while electrodes incorporated into the device signal that tissue contact is being made simultaneously both in the canal and on the external surface of the cervix.

13. a method to produce miniature electrode arrays by embedding printed circuit boards in plastic and forming into appropriate shapes for tissue analysis.

14. a method to ensure that patient tissue data is permanently linked to the patient identification through the use of a fingerprint reader to collect unique identification from the patient which is encoded with the patient tissue data collected.

15. a method to more accurately predict individualized patient risk using patient tissue maps and additional patient information correlated with increased risk of cancer.
